(19) United States
(12) Patent Application Publication
(10) Pub. No.: US 2001/0051790 A1
(43) Pub. Date: Dec. 13, 2001

(54) FINISHING TECHNIQUE FOR A GUIDING CATHETER

(76) Inventor: FRED T. PARKER, MONROE, IN (US)

Correspondence Address: SHASHANK S UPADHYE, PATENT ATTORNEY, P O BOX 2269, BLOOMINGTON, IN, 474022269

(21) Appl. No.: 09/464,096

(22) Filed: Dec. 16, 1999

Related U.S. Application Data
(60) Provisional application No. 60/112,491, filed on Dec. 16, 1998.

(51) Int. Cl. A61M 31/00; A61M 25/00

(52) U.S. Cl. 604/103.01; 604/524

(57) ABSTRACT

Disclosed is a catheter, more particularly a guiding catheter (10). The guiding catheter (10) comprises an inner layer (46), a reinforcement layer (24), and an outer layer (16). The reinforcement layer (46) generally extends from the proximal end (14) of the guiding catheter (10) and terminates proximal to the distal end (18) of the guiding catheter. The distal portion of the guiding catheter (10) comprises a soft material to facilitate manipulation. The reinforcement layer (24) can be attached to the inner layer (46) and the outer layer (16) attaches to both the reinforcement layer (24) and the inner layer (46). Proximal portion of the guiding catheter can have a folded cuff (52).

### CROSS-REFERENCE TO RELATED APPLICATION

This application claims priority of provisional application Serial No. 60/112,491 filed Dec. 16, 1998.

### TECHNICAL FIELD

The invention generally relates to a guiding catheter device and more particularly, to a guiding catheter having a tip, body, and reinforcement layer.

### BACKGROUND OF THE INVENTION

Without a doubt, the role of catheters played and continues to play an important role in interventional medicine. Catheters permit physicians to perform traditionally invasive procedures in a relatively non-invasive manner. To this end, development in catheter technology is an on-going project.

Various devices are used in catheter oriented procedures. For example, in percutaneous vascular access in cardiac intervention, a physician will first locate the femoral artery. Once located, small skin incisions are made two to three centimeters below the inguinal ligament. The femoral artery is located and supported by the fingertips of the physician. A needle is used to puncture the artery, that is, the distal end of the needle is inserted into the lumen of the artery. The distal end of the needle normally is not further advanced to puncture the distal wall of the artery, thereby maintaining the distal end of the needle within the lumen of the artery. The needle is stabilized and the physician then introduces a guidewire into the cannula of the needle and subsequently advancing the guidewire into the lumen of the artery. The distal tip of the guidewire is advanced into the lumen such that sufficient guidewire is in the lumen and will not be squirted out of the vessel due to the pulsatile nature of the vessel. The proximal end of the guidewire is located outside the body. The needle is then slid off the guidewire and removed from the procedure.

The guidewire now can act as a guide for guiding catheters. Guiding catheters are usually designed to meet several functions. First, the guiding catheter permits access to the coronary ostium and in this regard, therefore, serves as a delivery conduit for other interventional devices, such as a balloon catheter. Second, the guiding catheter provides support to the additional interventional device for advancement and deployment. Third, the fluid pressure in the lumen of the guiding catheter or at the catheter tip can be measured using standard pressure transducers. Finally, the guiding catheter can present visualization of the arterial tree by the release of standard radiographic contrast agents into the artery, or by the guiding catheter itself.

The support provided by the guiding catheter can be inherent or active. Inherent support for the guiding catheter originates, in one aspect, from the stiffness of the materials used in its construction. In addition, inherent support derives from the actual shape of the guiding catheter, notably from the tip configuration. On the other hand, active support of the guiding catheter typically is achieved either by manipulation of the guiding catheter itself or from the combined shape of the guiding catheter in relation to the targeted area, for example, the aortic arch.

As such, the guiding catheter is generally designed with several considerations in mind. First, several desirable features should be considered, such as, but not limited to, a large internal lumen, increased radial strength (to minimize the potential for kinking or collapse), low frictional resistance (both internal and external), columnar and torsional rigidity (to augment or enhance pushing force or torque), flexibility, malleability, and radiopacity. As is seen though, several features mutually depend on each other. For example, to increase the guiding catheter lumen diameter, a compromise is struck by decreasing the guiding catheter wall thickness, which then compromises radial strength, torsional rigidity, and the overall stiffness. This results in decreased desirable manipulation of the guiding catheter. To counter this, however, guiding catheters can have several composite features such as a softer, more malleable tip (located approximately in the distal eight to ten centimeter range) to enhance its utility and movement with the remaining portion of the guiding catheter being harder to provide the necessary torsional rigidity and structural integrity. This is generally achieved by using a material that is of higher durometer in the guiding catheter body than the distal tip section.

The ingredients used in fabricating the guiding catheter also play an important role in achieving the desired features. Typically, a guiding catheter comprises three layers: an inner tubular layer, a middle or intermediate layer, and an outer layer. The inner tubular layer typically comprises an inert, lubricious, biocompatible material selected to limit or minimize the potential for thrombosis and to minimize the frictional resistant force associated with the passage of another catheter or device through the lumen of the guiding catheter. In addition, it is also typical, but not required, to line the lumenal wall of the guiding catheter to further facilitate movement and reduce friction. For example, polytetrafluoroethylene, such as TEFLON®, can be used to line the lumenal surface as it provides superior performance over unlined guiding catheters.

The middle or intermediate layer usually comprises a heavier stock material to provide resistance to deformation, kinking, or collapse. This layer is typically made of stainless steel or KEVLAR® and can be formed into a weave or braid configuration, but is not limited to these configurations. Depending on the desired features of the guiding catheter, the middle layer can begin at the proximal end of the guiding catheter and terminate somewhere in the distal portion of the guiding catheter.

The outer layer generally comprises another material that is lubricious, biocompatible, and non-thrombogenic, such as polyethylene or polyurethane, or some combination thereof. In addition, it is desirable that the outer surface of the outer layer be smooth as to minimize trauma to the intimal wall of the vessel, such as causing abrasion to the wall, dislodging plaque, puncturing the vessel wall, or causing embolisms. The outer layer, middle layer, and inner tubular layer can also be fitted with a radiopaque marker to increase radiographic visualization.

As mentioned above, the inner tubular layer is generally a liner.

However, often times during the fabrication of the guiding catheter or during subsequent use, the liner can become loose, fray, and catch on anything being passed through the guiding catheter lumen. More urgently, the loose material can completely separate thus traveling through the vessel posing hazard to the patient. Thus, further improvements in the guiding catheter technology that secures the liner are well received.

In addition, during fabrication the middle layer can pose problems.

Desirably, the middle layer comprises a high tensile wires for the braid (440,000 psi and higher). In this manner, using high tensile wires gives the guiding catheter better torque and minimizes kinking in the guiding catheter. The higher the tensile wire used in the braiding indicates a greater propensity for unraveling. Thus, technology advances that secure the braiding also are well-received.

Furthermore relating to the distal tip fabrication, prior guiding catheters reveal a problem associated with distal tip dislocation or detachment. Desirably, the distal tip comprises a softer material than that of the outer layer. This design facilitates the movement of the distal tip and hence the guiding catheter into delicate and tortuous vessels, such as the coronary arteries. However, in constructing softer distal tips, often times the distal tip is prefabricated and then bonded to the guiding catheter body. A problem with this design is that the contact surface between the distal tip and the guiding catheter body is abrupt and thus subject to disconnection or detachment. Therefore, a technical advance that minimizes or eliminates the propensity of distal tip disconnection is desirable.

### SUMMARY OF THE INVENTION

The foregoing problems are solved and a technical advance is achieved in an illustrative guiding catheter. The present invention generally relates to a guiding catheter having a distal tip, a reinforcement layer, an inner tubular layer, and an outer body. The fabrication of the guiding catheter provides a better connection of a distal tip to the guiding catheter body. In addition, the present invention provides a better method of securing a reinforcement layer to the guiding catheter and the inner tubular layer such that ends of the reinforcement layer do not protrude out of the guiding catheter body nor cause trauma to the intimal wall.

For example, the guiding catheter can be constructed as to provide reinforcement to the distal tip so that dislocation and disconnection is minimized. In addition, the proximal end of the guiding catheter may be provided with a cuff to tighten the inner layer.

### BRIEF DESCRIPTION OF THE DRAWINGS

FIG. 1 demonstrates a side view of the invention.

FIG. 2 demonstrates a side view of the distal portion of the invention.

FIG. 3 and 3B demonstrate a cross sectioned side view of the invention.

FIG. 4 demonstrates a side view of the proximal portion of the invention.

FIG. 5 demonstrates a sectioned view of the proximal portion of the invention.

FIG. 6 demonstrates another embodiment of the proximal portion of the invention.

FIG. 7 demonstrates yet another embodiment of the proximal portion of the invention.

### DETAILED DESCRIPTION OF THE INVENTION

In accordance with the present invention, the following non-limiting examples are shown. FIG. 1 depicts a partial cross-section of a guiding catheter 10. Generally, guiding catheter 10 comprises a handling portion 12 located in the proximal end 14 of the guiding catheter 10. From the handling portion 12, extends a body 16 of the guiding catheter 10, which can extend for a specified length, such as over 100 centimeters. The length of the body 16 generally depends on the desired use of the guiding catheter 10 and the desired distance the guiding catheter 10 must travel to the situs. Guiding catheter 10 also has a distal end 18 of the guiding catheter, which terminates into a distal tip 20. Throughout the body 16 is a guiding catheter lumen 22, which extends between the proximal end 14 and the distal end 18. The outer diameter of lumen 22 is preferably maximized in relation to the outer diameter of the body 16. In this manner, the guiding catheter 10 can accommodate a larger catheter therein without significantly increasing the outer diameter of the body 16 such that the guiding catheter 10 is of limited utility in that it cannot be placed into smaller vessels, cavities, or the like. Various instruments can be introduced into the guiding catheter 10 via the handling portion 12 and inserted into the guiding catheter lumen 22.

With regard to its construction, guiding catheter 10 should be made of a biocompatible material to reduce the risk of complications during interventional procedures. For example, the body 16 can comprise a lubricious, biocompatible, non-thrombogenic material such as polyethylene, polyurethane, or some combination thereof. Furthermore, desirably body 16 is smooth, uniform, and without seams or abrupt transitions. This renders the body 16 nearly non-traumatic to the vessel or cavity wall. The material used to construct the body 16 can be specifically selected based on the intended durometer of the material. Where the guiding catheter 10 is intended for uses in smaller, tortuous vessels, desirably guiding catheter 10 and hence body 16 can be of lower durometer thus permitting more flexibility but be of sufficient strength to withstand the pushing and kinking forces. The body 16 can be made of variable materials such that the durometer changes from the proximal end 14 to distal end 18.

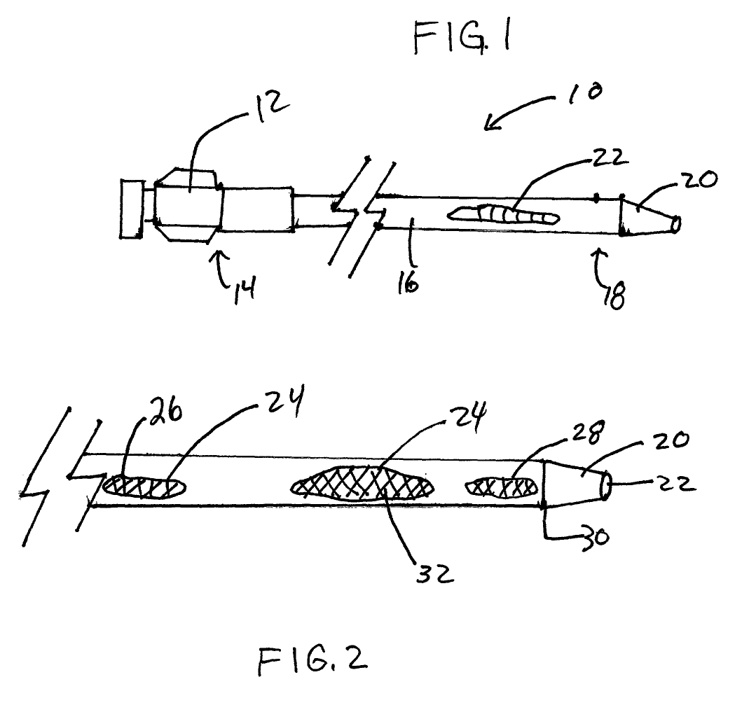

With respect to FIG. 2, shown is a sectioned view of the body 16. Disposed underneath the body 16 is a reinforcement layer 24, which itself has a reinforcement proximal end 26 and a reinforcement distal end 28. Reinforcement layer 24 generally is disposed under the body 16 and in some embodiments, the reinforcement layer 24 can be shorter than the body 16, that is, reinforcement layer 24 can terminate proximal to the distal end of the body 16. A radiopaque marker 30 can be located generally at the reinforcement distal end 28, the distal end of the body 16, or at the distal tip 20. Radiopaque marker 30 comprises a material sufficient to render the distal end 18 radiographically visible during the medical procedures. For example, the body, catheter, tip, or reinforcement layer can be made entirely or partially radiopaque. The radiopaque marker 30 can comprise a dense material such as tantalum, bismuth, barium, tungsten, or some combination thereof. Preferably, the radiopaque marker 30 is 80 percent tungsten and 20 percent nylon. Radiopaque marker 30 can be disposed over the reinforcement layer 24 such that the radiopaque marker 30 terminates proximate with the end of reinforcement distal end 28. However, marker 30 can be disposed anywhere along the device.

The reinforcement layer 24 provides structural integrity, strength, and promotes torqueability and minimizes the kinking of the guiding catheter 10. In this manner, the reinforcement layer 24 permits a stronger material to be used without significantly increasing the outer diameter of the body 16. The reinforcement layer 24 can comprise stainless steel, KEVLAR®, or other stronger material shaped into various configurations. Preferably, reinforcement layer 24 comprises stainless steel, such as but not limited to, being in the configuration of a braid 32. When using stainless steel as a braid 32, the tendency of the braid 32 to unwind increases due to the high tensile forces and thus braid 32 can be annealed in the reinforcement distal end 28 to form an annealed braid distal end 34. The braid 32 can be annealed using heat, which softens the metal and prevents the braid distal end 34 from flaring out after it is cut to shape. The annealing of braid 32 also prevents the braid 32 from unraveling and having rough edges at the braid distal end 34 that might puncture the body 16 and cause trauma to the vessel or cavity wall. Preferably, the braid distal end 34 is annealed for 1-5 cm and more preferably is annealed to a length of 1-3 cm. Preferably, braid distal end 34 terminates coincident with the radiopaque marker 30.

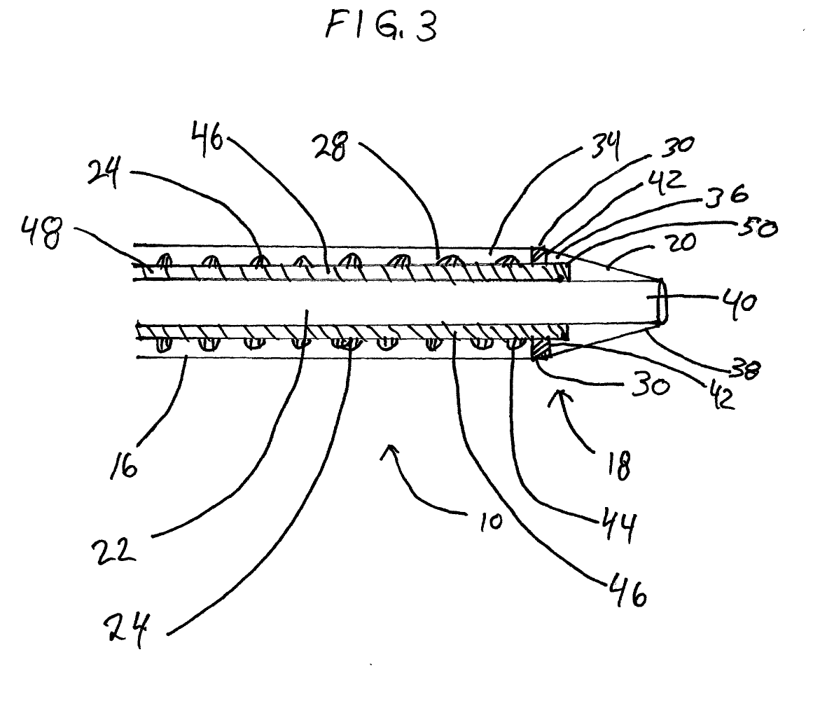

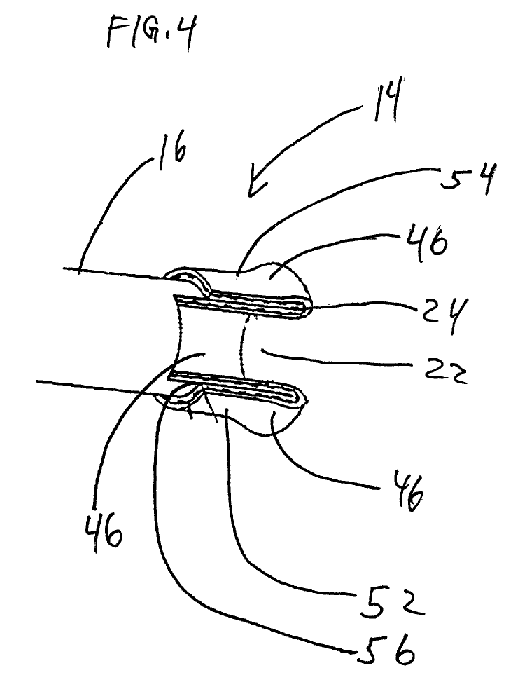

With reference to FIG. 3, shown is the distal end 18 of guiding catheter 10. In one embodiment of the present invention, radiopaque marker 30 and braid 32 terminate coincident with each other. Distal tip 20 comprises a tip proximal portion 36, a tip distal portion 38, and a tip lumen 40. The distal tip 20 and radiopaque marker 30 junction creates a distal tip-radiopaque marker interface 42, which demonstrates where the distal tip 20 joins the most distal part of body 16 and generally where the tip 20 begins to taper. Distal tip lumen 40 communicates with guiding catheter lumen 22 such that it provides a relatively smooth transition therebetween. Distal tip 20 can also be made of a radiopaque material to increase visualization.

Reinforcement layer 24 also includes a most distal end 44, which marks the termination of the reinforcement layer 24. As shown in FIG. 3, reinforcement layer 24 can extend to the interface 42. However, in other embodiments, the most distal end 44 can terminate before the interface 42.

Reinforcement layer 24 is disposed over an inner tubular layer 46, which includes an inner tubular proximal end 48 and an inner tubular distal end 50. Inner tubular layer 46 has a lumen 22. As shown in FIG. 3, inner tubular distal end 50 extends into the tip proximal portion 36. Preferably, inner tubular layer 46 can extend for a length of 1-2 mm into the tip proximal portion 36, however it can extend more into the tip proximal portion 36 as desired. Similarly, reinforcement layer 24 can extend for some distance into the proximal portion of the tip. Depending on the length of distal tip 20, the most distal end 44 of reinforcement layer 24 can also extend into the tip proximal portion 36. Preferably, the guiding catheter 10 is so constructed that the most distal end 44 terminates within 9 mm proximal to the inner tubular distal end 50. More preferred is that the most distal end 44 terminate within 5 mm of the inner tubular distal end 50 and most preferred is where the most distal end 44 terminates within 1 mm proximal to the inner tubular distal end 50. In one particular embodiment, as shown in FIG. 3, inner tubular distal end 50 extends only 1-2 mm into the tip proximal portion 36, thereby having the most distal end 44 of the reinforcement layer 24 terminate near the interface 42 and coincident with radiopaque marker 30. Thereby, reinforcement layer 24 is further secured in that radiopaque marker 30 sits atop reinforcement layer 24 and ensures that reinforcement layer 24 does not unravel or have protruding edges. Reinforcement layer 24 and most distal end 44 can terminate within 9 mm proximal of the interface 42, preferably within 5 mm proximal of the interface 42, and most preferably within 1 mm proximal thereof.

Inner tubular layer 46 comprises a biocompatible material that is strong enough to withstand the delivery of an instrument within the inner tubular layer lumen 22. For example, the inner tubular layer 46 can comprise a fluorocarbon, polyamide, polyolefin, polyimide, or some combination thereof. Preferably, inner tubular layer 46 comprises polytetrafluoroethylene .

In construction of the guiding catheter 10, the reinforcement layer 24 is disposed over the inner tubular layer 46. The body 16 is wrapped around the reinforcement layer 24—inner tubular layer 46 construct to form the guiding catheter 10. The distal tip 20 is slid adjacent to the body 16 by sliding it over the distal portion of the inner tubular layer 46 as shown in FIGS. 1, 2, 3 and 3B. The distal tip 20 can be provided with a tip recess 43 to accommodate the inner tubular layer 46 or reinforcement layer, or both, to facilitate the construction. Shrink wrapping is done by wrapping the body 16 and distal tip 20. The shrink wrapped guiding catheter 10 is then heated to melt the body 16, which generally comprises nylon tubing, so the body 16 material flows down through the lacunae of the braid 32 and mechanically bonds to the inner tubular layer 46 after the nylon cools. In addition, by selecting the materials desired, the radiopaque marker 30 and distal tip 20 can also melt in such a manner that the material of the distal tip 20 bonds to the inner tubular layer 46 below it and bonds to the radiopaque marker 30 adjacent to it. The shrink wrap is removed and the distal tip 20 is reheated over a mandrel projecting into the tip lumen 40 such that the distal tip 20 can be extended to a desired length and diameter. The distal tip 20 can also be shaped into various configurations. Thus, the shrink wrapping and heating process permits the body 16 to melt sufficiently to provide additional insurance that the reinforcement layer 24 will not easily unravel and ensures that the distal tip 20 will not dislodge or detach since the distal tip 20 is bonded to the underlying inner tubular layer 46 and to either the body 16 itself or the radiopaque marker 30, or both.

With reference to FIG. 4, inner tubular layer 46 can comprise a material that facilitates easy movement of a medical device within the lumen. In another embodiment of the present invention, the proximal end 14 of the guiding catheter 10 can be made to maximize the tension of the inner tubular layer 46 to prevent sagging or catching. As described above, inner tubular layer 46 can comprise a TEFLON® material. As such, devices inserted into the lumen 22 can catch, scrape, or tear at the inner tubular layer 46. Therefore, tightening the inner tubular layer 46 up against the reinforcement layer 24 and the body 16 minimizes this problem. Like any material, stretching it makes it less likely to sag and reduces the likelihood of something else catching on it. Body 16 has a proximal portion 14 where the body 16 inserts into the handling portion 12. By folding the body 16 proximal portion 1 4 inside out over the body 16 to form a cuff 52, the inner tubular layer 46 forms the cuff outside surface 54 of the cuff 52. The cuff inner surface 56 of the cuff 52 is the outside surface 58 of the body 16.

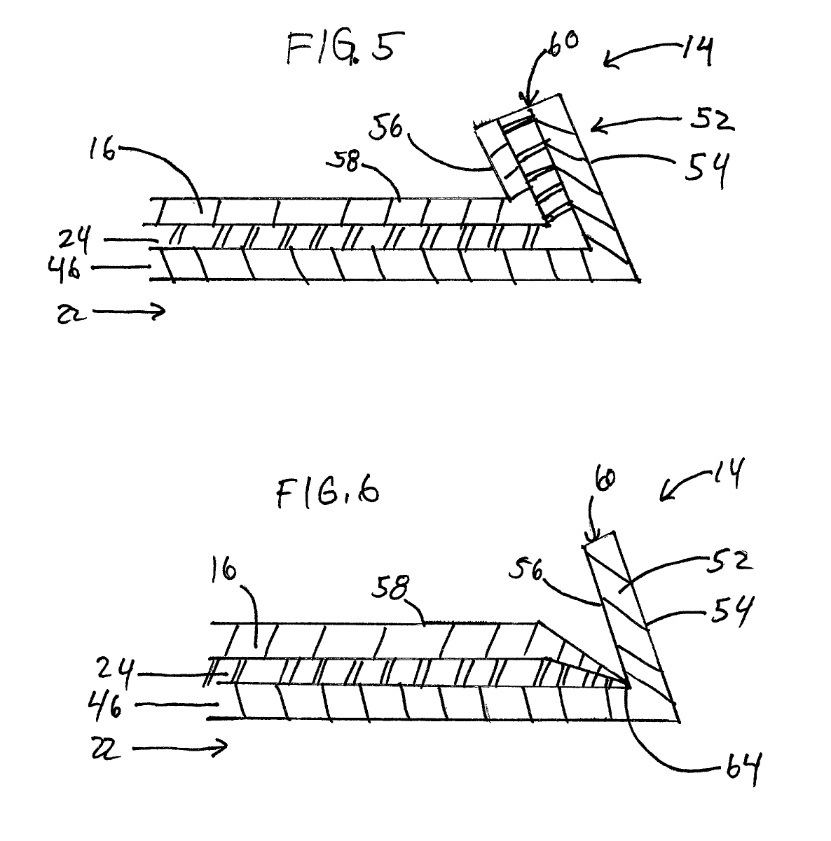

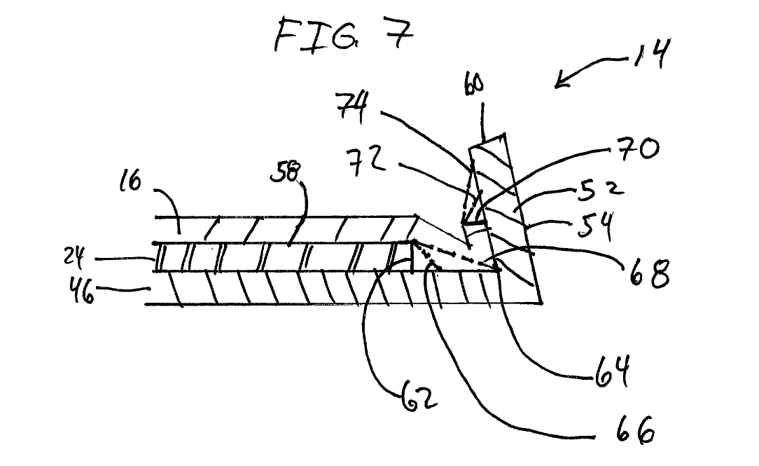

With reference to FIG. 5, shown is a section of the proximal portion 14 of the guiding catheter tube cut along its longitudinal axis and unfolded into a flattened multilayered structure. FIG. 5 demonstrates one aspect of cuff 52 formation having a cuff edge 60 that is the end most portion of the cuff 52. Body 16 has an outside surface 58. Although shown in FIG. 5, it is not necessary that reinforcement layer 24 terminate coincident with the body 16 or inner tubular layer 46 in the cuff 52, as the reinforcement layer 24 can terminate in the proximal portion 14 before the cuff 52 begins. Similarly, it is not necessary that the body 16 and inner tubular layer 46 terminate coincident at the cuff edge 60. As shown in FIG. 6, by having the relatively thicker body 16 or reinforcement layer 24 terminate before, or taper as they enter the cuff 52, the amount of material folded over into the cuff 52 is significantly reduced. As such, the cuff edge 60 will comprise a relatively thin layer of the inner tubular layer 46, of body 16, or reinforcement layer 24; or some combination thereof. Various embodiments are shown in FIGS. 6 and 7.

With respect to FIGS. 6 and 7, shown are various embodiments of the proximal portion 14 and cuff 52 demonstrating the various termination points of the reinforcement layer 24 and body 16. With respect to reinforcement layer 24, reinforcement layer 24 can terminate abruptly forming a reinforcement layer abrupt end 62, or taper such that it terminates prior to the cuff 52 and prior to cuff junction 64, which is the junction at which the cuff 52 begins to turn outward. Illustratively shown in FIG. 7, reinforcement layer 24 can terminate in a reinforcement layer medium taper 66, which terminates prior to the cuff junction 64, or can terminate in a reinforcement layer long taper 68, which terminates at the cuff junction 64. Reinforcement layer 24 can terminate in the cuff as it can terminate after the cuff junction 64.

Again with respect to FIG. 7, body 16 can terminate prior to, at, or after cuff junction 64. Illustratively shown, body 16 can terminate in a body abrupt end 70, in a body medium taper 72, which terminates prior to the cuff edge 60, or can terminate in a body long taper 74, which terminates coincident with cuff edge 60.

In this manner, the inner tubular layer 46 is pulled tightly against the reinforcement layer 24 and the body 16 due to the cuff 52 formation. The amount of material forming cuff 52 is significantly reduced if reinforcement layer 24 and body 16 taper prior to, or coincident with the cuff junction 64.

In yet another embodiment of the present invention, the reinforcement layer 24 and body 16 can be disposed over the inner tubular layer 46. In addition to the cuff 52 providing a snug, non-snagging surface, the inner tubular layer 46 could be further affixed to the reinforcement layer 24. Such means for affixing the inner tubular layer 46 to the reinforcement layer 24 or body 16, include but is not limited to, bonding, adhesion, mechanical attachment, heat sealing, compression, or other well-known ways to adhere one layer to another.

It is understood that the above described guiding catheter is merely an illustrative embodiment of the principles of the disclosed invention. As such, other embodiments of the invention are contemplated as identified and protected within the appended claims.

What is claimed is:

1. A guiding catheter, having a proximal end and a distal end, the guiding catheter having a distal tip proximate to the distal end of the guiding catheter, and the distal tip having a proximal portion, a distal portion and a first lumen extending therebetween, comprising:
a) an inner tubular layer having a proximal end, a distal end, and a second lumen extending therebetween, the second lumen communicating with the first lumen, the distal end of the inner tubular layer extending into the proximal portion of the distal tip;
b) a radiopaque marker disposed on the inner tubular layer proximate to the distal end of the inner tubular layer; and
c) a reinforcement layer having a proximal end and a distal end, the reinforcement layer being disposed over the inner tubular layer and the distal end of the reinforcement layer terminating proximate with the radiopaque marker.

2. The guiding catheter of claim 1 wherein the inner tubular layer further comprises a biocompatible material of at least one of a fluorocarbon, polyamide, polyolefin, and polyimide.

3. The guiding catheter of claim 2 wherein the inner tubular layer comprises polytetrafluoroethylene.

4. The guiding catheter of claim 1 wherein the reinforcement layer comprises a braid.

5. The guiding catheter of claim 4 wherein the braid comprises a stainless steel braid.

6. The guiding catheter of claim 5 wherein the braid comprises an annealed braid, the annealed braid having a distal end.

7. The guiding catheter of claim 6 wherein the braid comprises an annealed braid, having a distal end 1 to 3 cm long.

8. The guiding catheter of claim 6 wherein the distal end of the annealed braid terminates coincident with the radiopaque marker.

9. The guiding catheter of claim 1 wherein the reinforcement layer terminates 5 mm proximally from the distal end of the inner tubular layer.

10. The guiding catheter of claim 1 wherein the reinforcement layer terminates 1 mm proximally from the distal end of the inner tubular layer.

11. The guiding catheter of claim 1 wherein the reinforcement layer terminates coincident with the distal end of the inner tubular layer.

12. The guiding catheter of claim 1 wherein the reinforcement layer comprises an annealed braid terminating 5 mm proximally from the distal end of the inner tubular layer.

13. The guiding catheter of claim 1 wherein the reinforcement layer comprises an annealed braid terminating 1 mm proximally from the distal end of the inner tubular layer.

14. The guiding catheter of claim 1 wherein the radiopaque marker is disposed proximate to the distal end of the guiding catheter.

15. The guiding catheter of claim 1 wherein the radiopaque marker comprises a tungsten and nylon marker.

16. The guiding catheter of claim 1, wherein the proximal end of the guiding catheter comprises a cuff.

17. A guiding catheter, having a proximal end and a distal end, the catheter having a body, the guiding catheter having a distal tip proximate to the distal end of the guiding catheter, the distal tip having a proximal portion, a distal portion, and a first lumen extending therebetween, comprising:
a) an inner tubular layer having a proximal end, a distal end, and a second lumen extending therebetween, the second lumen communicating with the first lumen, the distal end of the inner tubular layer extending into the proximal portion of the distal tip;
b) a radiopaque marker disposed on the inner tubular layer proximate to the distal end of the inner tubular layer; and
c) a reinforcement layer having a proximal end and a distal end, the reinforcement layer being disposed over the inner tubular layer and the distal end of the reinforcement layer terminating coincident with the radiopaque marker; and
d) the reinforcement layer distal end extending into the proximal portion of the distal tip.

18. The guiding catheter of claim 17, wherein the reinforcement layer terminates within 9 mm proximal to the inner tubular layer distal end.

19. The guiding catheter of claim 17, wherein the reinforcement layer terminates within 5 mm proximal to the inner tubular layer distal end.

20. The guiding catheter of claim 17, wherein the reinforcement layer terminates within 3 mm proximal to the inner tubular layer distal end.

21. The guiding catheter of claim 17, wherein the reinforcement layer terminates near the radiopaque marker.

22. The guiding catheter of claim 17, wherein the radiopaque marker is secured to the distal tip.

23. The guiding catheter of claim 17, wherein the reinforcement layer is secured to the distal tip.

24. The guiding catheter of claim 17, wherein the proximal end of the guiding catheter comprises a cuff.

25. The guiding catheter of claim 24, wherein the cuff further comprises a fold of the proximal inner tubular end folded over at least one of the body and the proximal reinforcement end.

26. The guiding catheter of claim 24, wherein at least one of the inner tubular layer and the reinforcement layer extends to an edge that comprises a taper at the cuff.

27. A guiding catheter, the guiding catheter having a proximal portion that comprises a cuff.

28. The guiding catheter of claim 27, wherein the cuff further comprises a fold of a inner tubular layer folded over at least one of a body and a reinforcement layer.

29. The guiding catheter of claim 27, wherein the cuff has a fold to minimize cuff thickness.
